(19) United States
(12) Plant Patent Application Publication
(10) Pub. No.: US 2015/0305218 P1
(43) Pub. Date: Oct. 22, 2015

(54) Petunia plant named 'KLEPH13235'

(50) ;

(71) Applicant: Klemm+Sohn GmbH & Co. KG, Stuttgart (DE)

(72) Inventors: Nils Klemm, Stuttgart (DE); Antonella Capo, Latina (IT)

(73) Assignee: Klemm+Sohn GmbH & Co. KG, Stuttgart (DE)

(21) Appl. No.: 14/120,062

(22) Filed: Apr. 21, 2014

(51) Int. Cl. A01H 5/00

(52) U.S. Cl. PLT/356.2

(57) ABSTRACT

A new Petunia plant particularly distinguished by light violet, double flowers, a medium form, and a mounding and upright plant habit, is disclosed.

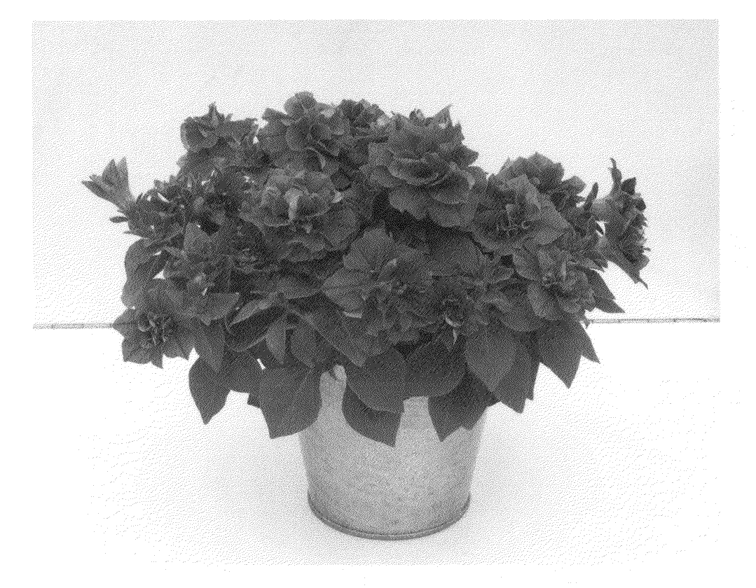

### GENUS AND SPECIES

Petunia hybrida

### VARIETY DENOMINATION

‘KLEPH13235 ’

### BACKGROUND OF THE NEW PLANT

The present invention comprises a new and distinct variety of Petunia, botanically known as Petunia hybrida, and hereinafter referred to by the variety name ‘KLEPH13235’. ‘KLEPH 13235’ originated from a cross-pollination conducted in July 2009 in Latina, Italy between the proprietary female Petunia variety ‘PH-2008-0221’ (unpatented) and the male Petunia variety ‘PH-2009-0893’ (unknown).

The seeds from the cross were sown and plants were grown in a greenhouse for evaluation, where an individual plant designated ‘KLEPH13235’ was selected from the group in April 2010 of plants in Latina, Italy having light violet, double flowers. In April 2010, ‘KLEPH 13235’ was first vegetatively propagated by terminal tip cuttings and tissue culture. ‘KLEPH13235’ was found to reproduce true to type in successive generations of asexual propagation via tissue culture and terminal tip cuttings.

### SUMMARY

The following are the most outstanding and distinguishing characteristics of this new variety when grown under normal horticultural practices in Stuttgart, Germany and Latina, Italy.

1. Light violet, double flowers;

2. A medium form; and

3. A mounding and upright plant habit.

### CROSS REFERENCE TO RELATED APPLICATIONS

Plant Breeders Rights for this variety have been filed with the European Community Plant Variety Rights Office on Feb. 2, 2014, Application No. 2014/0337 and in Canada on Feb. 18, 2014, Application No. 14-8213. ‘KLEPH13235’ has not been sold or made publicly available more than one year prior to the filing of the instant application.

### DESCRIPTION OF THE PHOTOGRAPH

This new Petunia plant is illustrated by the accompanying photograph of a plant grown in a glass greenhouse in Stuttgart, Germany. The photograph was taken in May 2013 and shows the foliage and flowers of a 3-month-old plant grown in a pot. The colors shown are as true as can be reasonably obtained by conventional photographic procedures.

### DESCRIPTION OF THE NEW VARIETY

The following detailed descriptions set forth the distinctive characteristics of ‘KLEPH13235’. The data which define these characteristics were collected from asexual reproductions carried out in Latina, Italy. Data was collected on plants grown 14-weeks from cultivation from potting in a plastic greenhouse in Latina, Italy in March 2014. Color references are to The R.H.S. Colour Chart of The Royal Horticultural Society of London (R.H.S.), 5^{th} edition (2007).
Classification:
   Family.—Solanaceae.
   Botanical.—Petunia hybrid.
   Common.—Petunia.
   Designation.—‘KLEPH13235’.
Parentage:
   Female parent.—The proprietary female Petunia variety ‘PH-2008-0221’ (unpatented).
   Male parent.—The proprietary male Petunia variety ‘PH-2009-0893’ (unpatented).
Plant:
   Form.—Medium.
   Habit.—Upright-mounding.
   Height (from top of soil).—15.0 cm.
   Width (horizontal plant diameter).—25.0 cm.
   Propagation.—Terminal tip cuttings and tissue culture.
   Time to produce a finished flowering plant.—14 weeks in the winter and 8 weeks in the spring.
   Time to initiate and develop roots.—2 to 3 weeks.
   Root description.—Moderate density, moderate branching, white roots.
Stems:
   Average number (basal).—18.
   Length of basal branches (from the base of the stem to the tip).—10.0 cm.
   Internode length.—0.5 cm to 1.0 cm.
   Diameter of branches (from midpoint).—0.4 cm.
   Stem color.—RHS 144B.
   Anthocyanin.—Absent.
   Texture.—Pubescent.
Leaves:
   Arrangement.—Alternate at the base of the stems, opposite on the flower stems.
   Length.—3.5 cm.
   Width.—3.0 cm.
   Shape.—Mature leaves are wide-obovate and immature leaves are elliptic.
   Apex.—Mature leaves are obtuse and immature leaves are acute.
   Base.—Attenuate.
   Margin.—Entire.
   Immature and mature leaf color.—Upper surface: RHS N137B Lower surface: RHS 137C.
   Texture (both upper and lower surfaces).—Slightly pubescent.
   Venation pattern.—Arcuate.
   Venation color.—Upper surface: RHS 146B Lower surface: RHS 144B.
   Petioles.—Length: 0.4 cm Diameter: 0.2 cm Color: RHS 144B Texture: Slightly pubescent.
Flower buds:
   Shape.—Irregular oblong.
   Length.—2.5 cm.
   Diameter.—0.8 cm.
   Color at tight bud.—RHS N92A and RHS 144A and RHS 144B.
   Texture.—Slightly pubescent.
Inflorescence:
   Blooming habit (flowering season).—Permanent flowering during summer time.
   Inflorescence type.—Cymose.
   Number of flowers per node.—1.
   Lastingness of individual blooms on the plant.—7 to 10 days.
   Fragrance.—Absent.
   Flowers.—Arrangement: Composed of 14 to 15 petals fused at the base Shape: Funnel-form Diameter (flower face): 5.5 cm Depth (total length of flower): 4.5 cm Throat/Funnel: Length: 2.5 cm Diameter (at opening): 1.2 cm Texture (both inner and outer surfaces): Smooth, glabrous Color, immature flower: Face/Margin color: RHS 83B Upper surface: RHS 83B Lower surface: RHS N82A and RHS N82B Color, mature flower: Face/Margin color: RHS 83B Upper surface: RHS 83B Lower surface: RHS N82A and RHS N82D Petals: Color: Inner surface: RHS 83B Outer surface: RHS N82A and RHS N82D Apex: Slightly mucronate Base: Irregularly fused Margin: Irregularly crenate Strength of waviness: Strong Degree of lobation: Weak Calyx arrangement: Actinomorphic, composed of 5 sepals Sepals: Color: Upper surface: RHSN N137B Lower surface: RHS 137C Length: 1.7 cm Width: 0.4 cm Shape: Oblanceolate Apex: Broadly acute Base: Attenuate Margin: Entire Texture (both upper and lower surfaces): Pubescent.
   Pedicels.—Color: RHS 144A Length: 2.5 cm Diameter: 0.25 cm Texture: Pubescent.
Reproductive organs:
   Stamens.—Quantity: 5 Shape: Needle with elliptic-ovate head Filament: Length: 1.5 cm Diameter: 0.05 cm Color: RHS 156A Anther: Shape: Round-ovate Color: RHS 4C Length: 0.25 cm Diameter: 0.15 cm Pollen: Amount: Variable.
   Pistils.—Quantity per flower: 1 Length: 0.7 cm Stigma color: RHS N82C Style color: RHS 1C.
Fruit and seed set: No fruit and seed set observed.
Disease and pest/insect resistance: No disease and pest/insect resistance observed.

### COMPARISON WITH PARENTAL LINES AND KNOWN VARIETY

‘KLEPH13235’ is a distinct variety of Petunia and is distinguished from its parents as described in Table 1.

TABLE 1
Comparison with Parental Lines

| | | Female Parent | Male Parent |
|---|---|---|---|
| Characteristic | ‘KLEPH13235’ | ‘PH-2008-0221’ | ‘PH-2009-0893’ |
| Flower color | Light violet | Salmon-pink | Dark pink-violet |
| Flower type | Double | | Single |

‘KLEPH13235’ is a distinct variety of Petunia and is most similar to the commercial Petunia variety ‘KLEPH09191’ (U.S. Plant Pat. No. 21,994), also commercially known as ‘Sweet Sunshine Burgundy’. Differences between the varieties are described in Table 2.

TABLE 2
Comparison with Similar Variety

| Characteristic | ‘KLEPH13235’ | ‘KLEPH09191’ |
|---|---|---|
| Flower color | Light violet | Purple-violet |

We claim:

1. A new and distinct variety of Petunia plant designated ‘KLEPH 13235’ as shown and described herein.
